(19) United States
(12) Patent Application Publication
(10) Pub. No.: US 2022/0257305 A1
(43) Pub. Date: Aug. 18, 2022

(54) BIPOLAR HF APPLICATOR AND HF APPLICATION SYSTEM

(71) Applicant: OLYMPUS WINTER & IBE GMBH, Hamburg (DE)

(72) Inventors: Thomas STEIN, Teltow (DE); Claudia Alexandra BOHNSACK, Hamburg (DE)

(73) Assignee: OLYMPUS WINTER & IBE GMBH, Hamburg (DE)

(21) Appl. No.: 17/671,654

(22) Filed: Feb. 15, 2022

(30) Foreign Application Priority Data
Feb. 15, 2021 (DE) 10 2021 103 506.6

(51) Int. Cl. A61B 18/14

(52) U.S. Cl. CPC A61B 18/14; A61B 2018/126

(57) ABSTRACT

A bipolar high-frequency (HF) applicator for an HF surgical instrument, and an HF application system are disclosed. The bipolar HF applicator has a flat main body that is made of an insulating material and has a rounded shape with two side faces opposite each other and an edge delimiting the side faces. The main body includes, on at least one of the two side faces, two electrodes, the electrode surfaces of which are isolated from each other on the side face. The electrodes are connected to supply lines for supplying HF energy.

### TECHNICAL FIELD

The present disclosure relates to a bipolar HF applicator for an HF surgical instrument as well as to an HF application system.

### BACKGROUND

Chronic rhinitis includes allergic rhinitis, nonallergic rhinitis and mixed sub-types. While the clinical manifestation can be different, watery rhinorrhea and nasal obstruction are dominating symptoms that negatively influence the quality of life of a patient and can cause him to seek treatment. Medication treatments for chronic rhinitis are considered the first choice. However, not all patients respond sufficiently to the available medication treatments and require in some circumstances procedural or surgical interventions for persistent chronic rhinitis.

In the past, neurectomy of the nervus vidianus was the method of choice for refractory, meaning therapy-resistant, rhinitis, while relatively newer technical studies have described the role of the posterior nasal neurectomy. Multiple systematic overviews that have recently been published have investigated the basis of evidence for the surgical treatment of chronic rhinitis. Although the vidian neurectomy appears to be effective, there are serious disadvantages, including the potential nasal and ocular morbidities and the increased healthcare costs and resources in connection with general anesthesia and surgical procedures.

The pathophysiology of chronic rhinitis is complex and includes both sensory and autonomous nerve pathways. Sensory nerve pathways detect specific allergens or irritants, which stimulate a parasympathetic reaction via the nervus vidianus. It has been shown that methods such as the vidian neurectomy reduce the symptoms of chronic rhinitis. However, side effects such as dry eyes due to the ablation of the parasympathetic innervation of the lacrimal gland have been found. The hypothesis has been proposed that the ablation of the posterior nasal mucous membrane or respectively the posterior nasal nerves (Rr. nasales posteriores) can reduce the side effects of dry eyes in a vidian neurectomy. Targeted therapies for this region that can offer relief of chronic rhinitis symptoms with limited side effects are therefore desired.

Due to its simple nature, cryotherapy has gained interest. In cryotherapy, liquid nitrogen is used to ablate posterior nasal tissue. Through extremely low temperatures, cryotherapy forms ice crystals and induces a cell contraction to then lyse the cells. An example of this is “ClariFix” by Arrinex, Inc. The cryotherapy device uses liquid nitrogen to generate very low temperatures. During the freezing process, the device does not receive a response regarding the tissue effect in order to adjust the temperature or the application time.

Another possibility to temporarily or permanently restrict the function of the posterior nasal nerve (PNN) is the use of radio frequency current. With this type of energy, the tissue is not frozen but heated. A known device for HF ablation of the posterior nasal nerve includes multiple wires as electrodes on the distal tip of an elongated insertion part.

### SUMMARY

The object of the present disclosure is to provide a bipolar HF applicator and an HF application system that allows a safe and efficient temporary or permanent restriction of the function of the posterior nasal nerve.

This object is solved by a bipolar HF applicator for an HF surgical instrument, with a flat main body made of an insulating material, that has a rounded, in particular round or elliptical, shape with two side faces opposite each other and an edge delimiting the side faces, wherein the main body has, on at least one of the two side faces, two electrodes, the electrode surfaces of which are isolated from each other on the side face, wherein the electrodes are connected to supply lines for supplying HF energy.

The bipolar HF applicator according to the present disclosure avoids the problems that arise in the use of thin wires as electrodes, as occurs in known devices for HF ablation of the posterior nasal nerve that include multiple wires as electrodes on the distal tip of an elongated insertion part. With thin electrode wires, a local dehydration of the tissue that is in contact with the electrode wires occurs. The dehydration leads to a reduction in the efficiency of the treatment in that the change of the impedance of the tissue ends the ablation early.

In contrast to this, the electrodes in the bipolar HF applicator according to the present disclosure are arranged on a side face or on both side faces of a flat main body made of an insulating material and occupy a configuration distributed over the side face, which leads to a significantly less localized energy input into the treated tissue as is the case with wired-shaped electrodes. Accordingly, the degree of dehydration is considerably lower and the treatment is more effective.

The HF applicator according to the present disclosure can be inserted through the nose into the nasal cavity and into the region of the posterior nasal nerve during an operation. Supplying HF energy causes heating of the tissue against which the electrodes of the HF applicator lie and to a sclerosing of the near-surface nerve tissue. The applicator can be used in a very targeted manner and leads to less destruction of tissue than wire electrodes. The HF applicator can have a circular shape, but also an elliptical or other shape with rounded corners. The greatest extension of the HF applicator, for example its diameter, is in the range of several millimeters, in particular preferably between 2 and 12 mm, in particular between 3 and 8 mm.

The HF applicator according to the present disclosure has only two electrodes. These can be easily connected to a bipolar standard output socket of an electrosurgical generator. For optimum treatment results, the generator can have an impedance/resistance feedback function in order to give the surgeon a signal when the coagulation process is completed. However, it would also work with a standard bipolar low-voltage coagulation mode of an HF generator, as used for bipolar pincettes.

High-frequency (HF, also “radio frequency,” RF) is understood in the context of the present disclosure to mean a frequency range from 100 kHz to 50 MHz, in particular between 300 kHz and 4 MHz.

In embodiments, the supply lines run inside the main body and emerge at the edge of the main body. This facilitates a very compact and smooth configuration of the HF applicator. The supply lines can therefore be cast, for example, into the main body in a mold casting process during the manufacturing of the main body.

In embodiments, the flat main body with the electrodes is designed to be flexibly bendable. As a result, it can be inserted into the nasal cavities of the patient very easily and with very low risk of injury and can adapt to the relief of the surface to be characterized and also inserted into curved gaps of the nasal passages between the nasal conchae. Flexible electrodes can be designed, for example, as conductive, for example metallic, foils.

Suitable materials for the main body are, for example, plastics, ceramics, or silicones, the temperature stability of which is sufficient for the temperatures occurring during HF surgical procedures. Typical temperatures are approximately 80° C., so that a temperature stability of approx. 100° C. or above is favorable. Silicones, as well as soft plastics, are particularly suitable for flexible HF applicators due to their softness while ceramics and harder plastics can be used for more rigid HF applicators. The main bodies of the HF applicators are produced using methods, for example injection molding, that are proven and known for the respective materials.

In embodiments, the two electrodes are circular, annular, or elliptical, wherein one of the two electrodes is arranged as an inner electrode, in particular concentrically, inside the other electrode, wherein in particular both the outer electrode and the inner electrode are annular. The HF field between the two electrodes is then also annular and has an extent that prevents too strong of a localization and dehydration of the treated tissue.

In alternative embodiments, the two electrodes are arranged next to each other on the side face of the main body, in particular basically semicircularly with an insulating strip between the electrodes. This arrangement also effects a flat, less localized distribution of the HF field and an accordingly less pronounced dehydration of the treated tissue.

In other embodiments, both side faces of the main body each have electrode surfaces of both electrodes, so that one bipolar electrode arrangement results on each of the two side faces of the main body. In this manner, nerve tissue can be cauterized on both sides of a nasal passage and the treatment time can be shortened.

In one embodiment with a two-sided application, the main body is completely penetrated by the two electrodes, wherein the electrodes are each designed to penetrate the entire main body as a solid piece. This simplifies the contact and represents a robust type of an HF applicator.

Alternatively, in embodiments, the electrodes are each set into recesses in the side face or the side faces of the main body, wherein the electrode faces terminate in particular flush with the side face or the side faces of the main body.

A development of the HF applicator provides that the main body comprises a canal structure for a fluid cooling medium, which can be introduced into the main body from outside and discharged again. Such a cooling of the HF applicator serves to cool the mucous membrane against which the HF applicator lies during the procedure. The supplied HF energy heats the upper tissue layers consisting of mucous membrane and nerve tissue. Through the cooling, however, so much heat energy is in turn drawn out of the mucous membrane that the temperature in the mucous membrane does not exceed a harmful amount. The cooling effect, however, does not go so far into the nerve tissue that a cauterization or sclerosing is prevented there. Overall, treatment with a cooled version of the HF applicator according to the present disclosure is very gentle and efficient. The risk of subsequent adverse events is further prevented.

The object underlying the present disclosure is based is also solved by an HF application system with at least one surgical HF instrument, which is equipped with a previously described bipolar HF applicator according to the present disclosure, and an HF generator, which is designed to supply the HF applicator with HF energy.

The HF application system embodies the same features, advantages, and characteristics as the bipolar HF applicator according to the present disclosure in its various embodiments.

In embodiments, the system may also include a cooling device and a cooling circuit. The cooling device can be capable of cooling a fluid cooling medium in order to cool the HF applicator. The cooling circuit can be designed to introduce the fluid cooling medium into a channel structure of the flat main body of the HF applicator for cooling the HF applicator. The cooling circuit can also receive the cooling medium that is discharged from the channel structure of the HF applicator. This achieves gentle and efficient treatment with further reduced risk of unwanted injuries. Another method for the cooling is the use of one or more Peltier elements, the hot side of which is cooled via the cooling circuit.

Further features of the present disclosure will become evident from the description of embodiments according to the present disclosure, together with the claims and the appended drawings. Embodiments according to the present disclosure can fulfill individual features or a combination of several features.

Within the context of the disclosed features which are labeled with “in particular” or “preferably” are to be understood to be optional features.

### BRIEF DESCRIPTION OF THE DRAWINGS

The present disclosure is described below, without restricting the general idea of the disclosure, based on exemplary embodiments in reference to the drawings, whereby we expressly refer to the drawings with regard to the disclosure of all details according to the present disclosure that are not explained in greater detail in the text. In the drawings:

FIG. 1 shows a schematic depiction of a first exemplary embodiment of a bipolar HF applicator according to the present disclosure from various perspectives as well as a surgical HF instrument with a distally arranged HF applicator,

FIG. 2a) shows a schematic depiction of the operating principle of the HF applicator from FIG. 1,

FIG. 2b) shows a schematic depiction of the operating principle of a second exemplary embodiment of an HF applicator according to the present disclosure with additional cooling,

FIG. 2c) shows a schematic depiction of the field of operation of an HF applicator according to one of FIGS. 1, 2a), and 2b),

FIG. 3a) shows a depiction of a use of the HF applicator from FIG. 1,

FIG. 3b) shows a depiction according to FIG. 3a) with the HF applicator flipped over,

FIG. 4 shows a schematic depiction of a third exemplary embodiment of an HF applicator according to the present disclosure,

FIG. 5 shows a schematic depiction of a fourth exemplary embodiment of an HF applicator according to the present disclosure,

FIG. 6 shows a schematic depiction of a fifth exemplary embodiment of an HF applicator according to the present disclosure,

FIG. 7 shows a schematic cross-sectional depiction of a channel system of an HF applicator according to the present disclosure, and

FIG. 8 shows a schematic cross-sectional depiction of a branched channel system of an HF applicator according to the present disclosure.

### DETAILED DESCRIPTION

In the drawings, the same or similar elements and/or parts are, in each case, provided with the same reference numerals such that a repeated presentation is dispensed with in each case.

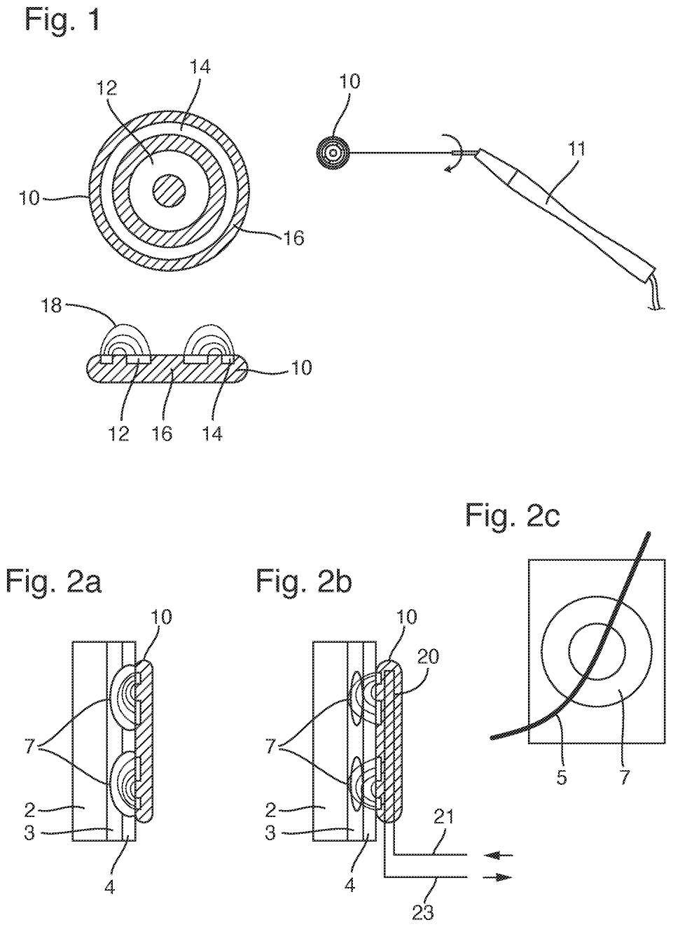

FIG. 1 schematically shows a first exemplary embodiment of a bipolar HF applicator 10 according to the present disclosure from various perspectives as well as a surgical HF instrument 11 with a distally arranged HF applicator 10. In the top view, the HF applicator 10 has a round shape with a circular main body 16, on the surface of which two concentric electrodes, namely an inner electrode 12 and an outer electrode 14, are arranged. The inner electrode 12 is designed in this exemplary embodiment as a circular ring, the center of which remains open. The material of the main body 16 can be seen here. In this exemplary embodiment, the width of the annular inner electrode 12 is greater than the width of the annular outer electrode 14. This results in that the surface areas of the two concentric electrodes 12 and 14 are approximately the same size. The difference is less than 20% in this exemplary embodiment. This results in a more uni-form field distribution in the inner region of the HF applicator 10.

In the lower part of FIG. 1, the HF applicator 10 is shown in cross-section. It is clear that it is a flat body, the upper and lower side faces of which are delimited toward the outside by a circumferential edge. The two electrodes 12, 14 are set into one of the two side faces, which is the upper side face in this depiction, in recesses and terminate in this exemplary embodiment flush with the upper side face of the main body 16. Field lines of the HF field 18 that is generated with the electrode pair of the HF applicator 10 are also shown. The cross-section through the HF field 18 shows that there is no HF field in the center, while the HF field formed between the inner and outer electrodes 12, 14 follows the annular concentric structure of the two electrodes 12, 14.

As shown in the upper and lower parts on the left half of FIG. 1, the flat main body 16 has a thickness extending in a direction between the two side faces, and a length and a width that are orthogonal to each other and define a plane that is orthogonal to the thickness and parallel to the two side faces. In particular, the upper part on the left half of FIG. 1 shows one of the two side faces, which extends in the length and width directions of the flat main body 16. Because, the flat main body 16 in FIG. 1 has a circular shape, the length and the width correspond to a radial direction of the circular flat main body 16. The lower part on the left half of FIG. 1 is a cross-sectional view showing the two side faces on opposite sides in the thickness direction. The length and the width of the flat main body 16 are larger than the thickness of the flat main body. Although the length and the width have the same dimensions in the circular embodiment shown in FIG. 1, the present disclosure is not limited to this. For example, as discussed below, the flat main body 16 may have an elliptical or oval shape, in which case the length may be larger than the width. In either case, the length and the width may each be larger than the thickness.

In the right half of FIG. 1, a surgical HF instrument 11 is shown, on the distal tip of which the bipolar HF applicator 10 is arranged. By means of an arrow, it is shown that the HF applicator 10 can be rotated about the connecting axis or respectively the connecting shaft between the hand part of the HF instrument 11 and the HF applicator 10. As shown in the right half of FIG. 1, the flat main body 16 is configured to be attached to the HF surgical instrument 11 such that the two side surfaces of the flat main body 16 extend parallel to a connecting shaft of the HF surgical instrument 11. In other words, the plane that is defined by the length and the width and is parallel to the two side faces of the flat main body 16 extends parallel to the connecting shaft of the HF surgical instrument 11, and the thickness of the flat main body 16 extends orthogonal to the connecting shaft of the HF surgical instrument 11. In some embodiments, in which the flat main body 16 has an elliptical or oval shape, the length or the width may extend in parallel to the connecting shaft of the HF surgical instrument 11.

FIG. 2a) shows a schematic depiction of the operating principle of the HF applicator 10 according to the first exemplary embodiment shown in FIG. 1. The HF applicator 10, which is supplied with HF energy and accordingly generates a characteristic HF field, lies directly against a layer structure of various tissue types in the posterior nasal cavity region. From inside to outside, it is a bone layer 2, followed by a nerve tissue layer 3, which can contain, for example, the nervus/ramus nasalis posterioris to be cauterized, followed by a mucous membrane 4, which lies above and protects the nerve tissue layer 3. The coagulation region, which is generated by the HF field of the HF applicator 10, is referred to with the reference sign 7. It penetrates through the mucous membrane 4 into the nerve tissue layer 3 and leads to a sclerosing of the contained nerve tissue there. The coagulation region 7, however, is not limited to the nerve tissue layer 3, but also comprises the mucous membrane 4.

FIG. 2b) shows the operating principle of a second exemplary embodiment of an HF applicator 10 according to the present disclosure with additional cooling. A channel system 20 for a fluid coolant, which is supplied and discharged via a supply line 21 and a discharge line 23, passes through the main body of the HF applicator 10 of the second exemplary embodiment. The coolant ensures that the main body of the HF applicator 10 is cooled, which in turn cools the mucous membrane 4 in the region in which the HF applicator 10 lies against the mucous membrane 4. This cools the mucous membrane 4 so much that the HF field penetrating the mucous membrane 4 no longer damages or coagulates the mucous membrane. The coagulation region 7 is limited to the nerve tissue layer 3 by this measure. The surface of the affected tissue is thus protected and is less susceptible to subsequent complications or infections.

Instead of a pure fluid cooling, a cooling element with one or more Peltier elements can also be used, the warm side of which must in turn be cooled. An advantage of this measure is a faster response of the cooling when it is required. It must be ensured in this case that the rear side does not become too hot, since the waste heat may only be transported away with a delay.

FIG. 2c) schematically shows the field of operation of an HF applicator 10 according to one of FIGS. 1, 2a), and 2b). A cut-out of the surface of the treated tissue in the nasal cavity is shown, through which runs a nerve 5. The coagulation region 7 is annular in this view, corresponding to the configuration of the HF field of the concentric electrode pair 12, 14. This has the advantage that the nerve 5 is severed at two positions, which ensures a shutdown of this nerve 5. At the same time, a more gentle application of HF energy into the tissue can take place with simultaneously secure shutdown of the nerve 5, as a result of which the tissue overall is less damaged.

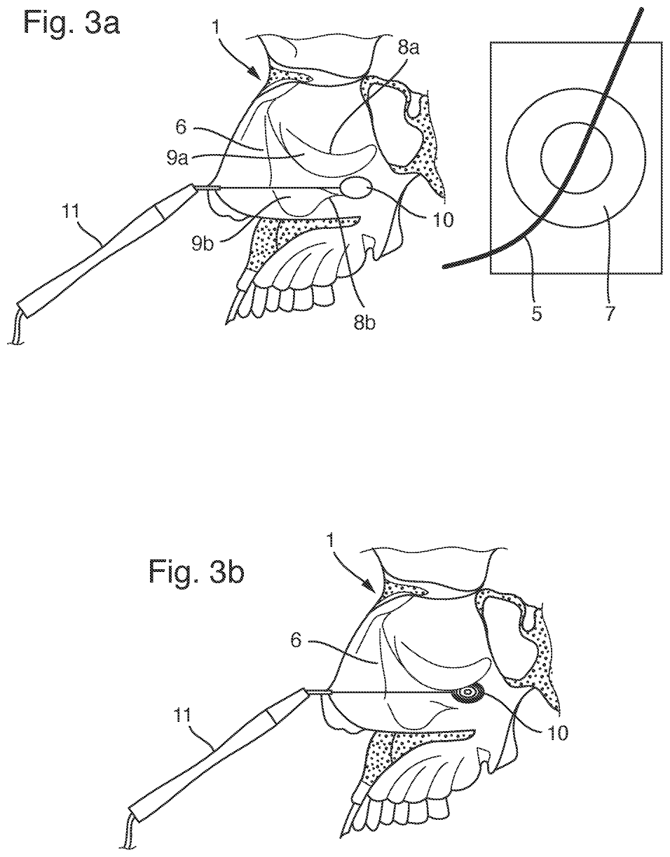

A section through the nasal cavity 6 of a human skull 1 is shown in FIGS. 3a) and 3b). In the depiction, the middle nasal concha 9a (concha nasalis media), the inferior nasal concha 9b (concha nasalis inferior), and the nerve branches rami nasales posteriores superiores laterales 8a and rami nasales posteriores inferiores 8b running on these two nasal conchae 9a, 9b are provided with reference signs.

The HF applicator 10 on the distal tip of an HF instrument 11 is inserted into the nasal cavity 6. The positioning of the HF instrument 11 is to be taken schematically, since the insertion takes place through the nostril (apertura nasi) and not through the nasal tissue. The HF applicator 10 is located in the nasal passage between the middle nasal concha 9a and the inferior nasal concha 9b. In this position, the lower nerve branch 8b should be ablated or temporarily or permanently hindered in its function in order to treat refractory rhinitis.

The applicator 10 is placed on the nervus/ramus nasalis posteriores 8b on the inferior nasal concha 9b as shown on the right in FIG. 3a). After reaching the correct positioning, HF energy is supplied and the nerve 5 is sclerosed in that the nerve tissue at this point is cauterized in an annular manner. Then, as shown in FIG. 3b), the HF applicator 10 can be rotated by 180° so that its active side faces the middle nasal concha, and nerve tissue in the middle nasal concha can also be sclerosed or respectively cauterized. The HF applicator 10 can be provided with cooling in accordance with FIG. 2b) in order to protect the mucous membrane at this point.

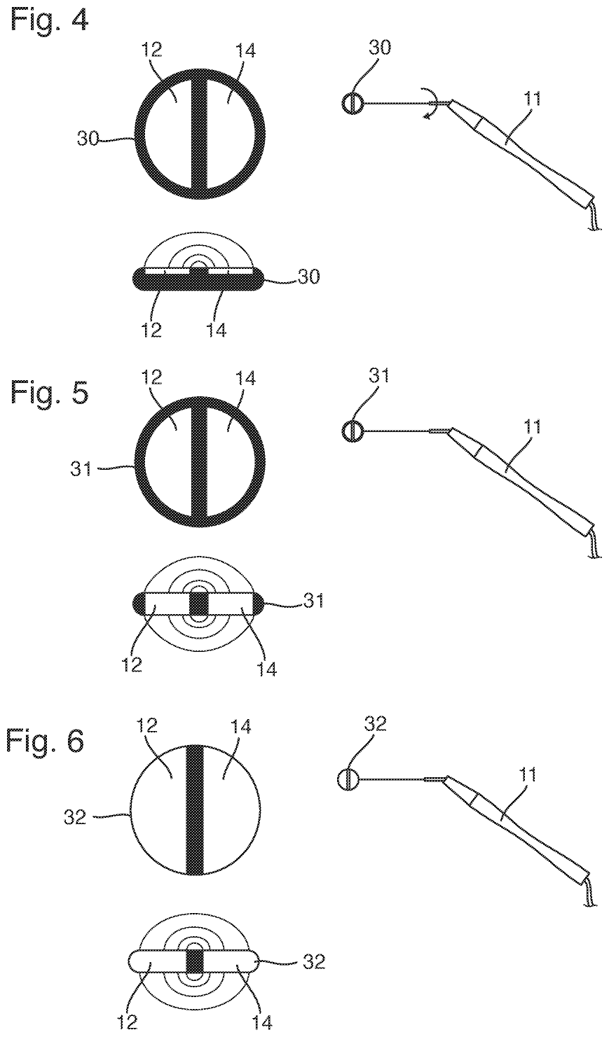

FIGS. 4, 5, and 6 show further exemplary embodiments of bipolar HF applicators 30, 31, and 32 according to the present disclosure. These differ from the first two exemplary embodiments of an HF applicator 10 in the configuration of the two electrodes 12, 14. In the further exemplary embodiments, they are each semicircular electrodes separated from each other by a nonconductive strip made of the material of the main body.

In the third exemplary embodiment in FIG. 4, the two semicircular electrodes 12, 14 are accommodated in recesses on one of the two side faces of the flat main body. This results in a flat, not very localized HF field whose vertical extent, which determines the depth of penetration into the tissue to be sclerosed, is greatest in the center. The electrodes 12, 14 are also surrounded by the insulating material of the main body on the circumferential edge.

The fourth exemplary embodiment of an HF applicator 31 of FIG. 5 differs from this in that the two electrodes 12, 14 are designed such that they penetrate the entire main body 16 and have electrode surfaces on both side faces of the main body 16. The HF field extends symmetrically to both sides of the HF applicator 31. This also applies to the HF applicator 32 of the fifth exemplary embodiment of FIG. 6, with the difference that the nonconductive material of the main body 16 in this case amounts to nothing more than the strip between the two electrodes 12, 14. This HF applicator 32 has no circumferential edge. The field distribution is therefore similar to that of the HF applicator 31 in FIG. 5, although distributed somewhat wider.

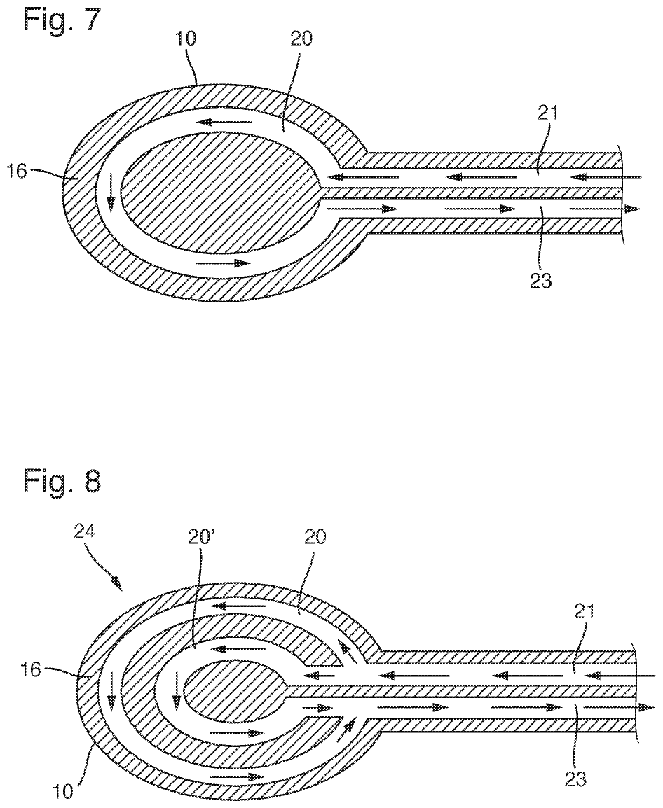

FIGS. 7 and 8 show two exemplary embodiments of channel systems 20 for cooling the HF applicator. In both cases, the basic shape of the main body 16 of the HF applicator 10 is elliptical. The channel structure 20 shown in FIG. 7 for a coolant, the flow of which through the channel structure 20 is shown with arrows, is of simple construction. It comprises a channel describing an elliptical pathway that is concentric to the basic shape of the elliptical main body 16 of the HF applicator 10. This channel is fed by the supply line 21 and opens again into the discharge line 23 for fluid coolant. The positioning ensures a uniform cooling performance over the entire main body 16.

It is advantageous if the positioning of the channel guarantees uniform heat dissipation from both electrodes of the HF applicator 10. In the electrodes 12, 14 of the HF applicators 30, 31, and 32 of FIGS. 4, 5, and 6, this is achieved, for example, by arranging them symmetrically about the midpoint of the respective HF applicator 30, 31, 32 just like the channel of the channel structure 20 in FIG. 7. Regarding the concentric electrode arrangement of the HF applicator 10 from FIGS. 1 and 2a, 2b, a favorable arrangement of such a channel is in the space between the two concentric electrodes 12, 14, possibly with an overlap with the two electrodes 12, 14 in the radial direction.

FIG. 8 shows an alternative with a branched channel structure 24, in which the fluid coolant in the supply line 21, after entering the main body 16 of the HF applicator 10, reaches a junction, in which a part of the coolant enters an outer channel of the channel structure 20 and another part continues to flow in an inner channel 20′. Both channel structures describe concentric elliptical pathways. Symmetrically with respect to the first junction, the outer channel structure 20 rejoins the inner channel 20′ after circumnavigating the base body 16 before merging into the discharge 23 downstream of the junction.

This branched channel structure 24 ensures an even stronger discharge of heat energy than the unbranched channel structure 20, because the heat has a short path to the nearest channel at every point in the main body 16.

The concentric channels are particularly efficient when, in the case of a concentric electrode arrangement like those of FIGS. 1 and 2, they are each arranged below the respective inner and outer diodes. These are metallic and thus good conductors of heat and cold. Due to their close proximity to the cooling channels, these can therefore support and reinforce the cooling effect.

All of the indicated features, including those which are to be inferred from the drawings alone, as well as individual features which are disclosed in combination with other features, are deemed to be essential to the present disclosure both alone and in combination. Embodiments according to the present disclosure can be fulfilled by individual features or a combination of several features.

### List of Reference Signs

1 Skull

2 Bone layer

3 Nerve tissue layer

4 Mucous membrane

5 Nerve

6 Nasal cavity

7 Coagulation region

8a Rr. nasales posteriores superiores laterales

8b Rr. nasales posteriors inferiors

9a Concha nasalis media

9b Concha nasalis inferior

10 HF applicator

11 Surgical HF instrument

12 Electrode

14 Electrode

16 Main body

18 HF field

20 Channel structure for fluid coolant

20′ Inner channel for fluid coolant

21 Supply line for fluid coolant

23 Discharge line for fluid coolant

24 Branched channel structure for fluid coolant

30 HF applicator

31 HF applicator

32 HF applicator

1. A bipolar high-frequency (HF) applicator for an HF surgical instrument, comprising:
a flat main body that is made of an insulating material, and has a rounded shape with two side faces opposite each other and an edge delimiting the side faces, the flat main body including, on at least one side face of the two side faces, two electrodes including electrode surfaces that are isolated from each other on the at least one side face,
wherein the electrodes are connected to supply lines for supplying HF energy.

2. The bipolar HF applicator according to claim 1, wherein the flat main body has a circular or elliptical shape.

3. The bipolar HF applicator according to claim 1, wherein the supply lines run inside the flat main body and emerge at the edge of the flat main body.

4. The bipolar HF applicator according to claim 1, wherein the flat main body with the electrodes is designed to be flexibly bendable.

5. The bipolar HF applicator according to claim 1, wherein:
the two electrodes are circular, annular, or elliptical, and
the two electrodes include an outer electrode and an inner electrode that is disposed inside the outer electrode.

6. The bipolar HF applicator according to claim 5, wherein the inner electrode and the outer electrode are annular, and the inner electrode is disposed concentrically inside the outer electrode.

7. The bipolar HF applicator according to claim 1, wherein the two electrodes are arranged next to each other on the at least one side face of the flat main body with an insulating strip between the two electrodes.

8. The bipolar HF applicator according to claim 7, wherein the two electrodes each have a semicircular shape.

9. The bipolar HF applicator according to claim 1, wherein the electrode surfaces of the two electrodes are exposed on the two side faces of the flat main body so that each of the two side faces of the flat main body includes a bipolar electrode arrangement.

10. The bipolar HF applicator according to claim 9, wherein the flat main body is completely penetrated by the two electrodes in a thickness direction extending between the two side faces of the flat main body.

11. The bipolar HF applicator according to claim 1, wherein the two electrodes are each set into recesses in the at least one side face of the flat main body, and the electrode surfaces are flush with the at least one side face of the flat main body.

12. The bipolar HF applicator according to claim 1, wherein the flat main body comprises a channel structure for a fluid coolant, the channel structure being configured to receive the fluid coolant into the flat main body from outside and discharge the fluid coolant from the flat main body.

13. The bipolar HF applicator according to claim 12, wherein the channel structure is branched within the flat main body such that two or more channels branch off from a fluid coolant supply line and a fluid coolant discharge line.

14. The bipolar HF applicator according to claim 12, wherein the flat main body has a circular or oval shape, and the channel structure has one or more circular or oval channels.

15. The bipolar HF applicator according to claim 14, wherein the channel structure has at least two circular or oval channels that are arranged concentrically to each other.

16. An HF application system comprising:
at least one surgical HF instrument, which is equipped with the bipolar HF applicator according to claim 1, and
an HF generator, which is designed to supply the HF applicator with HF energy.

17. The HF application system according to claim 16, further comprising:
a cooling device that is configured to cool a fluid cooling medium, and
a cooling circuit that is configured to:
introduce the fluid cooling medium into a channel structure of the flat main body of the HF applicator for cooling the HF applicator, and
receive the fluid cooling medium that is discharged from the channel structure of the HF applicator.

18. A bipolar HF applicator for an HF surgical instrument comprising:
a flat main body that is made of an insulating material, and includes two side faces that have a round or elliptical shape and are opposite each other, and an edge delimiting the two side faces, the flat main body having a length, a width, and a thickness, the thickness extending in a direction between the two side faces, the length and the width being orthogonal to each other and defining a plane that is orthogonal to the thickness and parallel to the two side faces, the flat main body including, on at least one side face of the two side faces, two electrodes including electrode surfaces that are isolated from each other on the at least one side face,
wherein the electrodes are connected to supply lines for supplying HF energy.

19. The bipolar HF applicator according to claim 18, wherein the flat main body is configured to be attached to the HF surgical instrument such that the length of the flat main body extends parallel to a connecting shaft of the HF surgical instrument.

20. The bipolar HF applicator according to claim 18, wherein the length and the width are larger than the thickness of the flat main body.
